(19) United States
(12) Patent Application Publication
(10) Pub. No.: US 2011/0110975 A1
(43) Pub. Date: May 12, 2011

(54) INACTIVATED VIRUS COMPOSITIONS AND METHODS OF PREPARING SUCH COMPOSITIONS

(75) Inventors: James A. Grunkemeyer, Omaha, NE (US); Wayne L. Ryan, Omaha, NE (US)

(73) Assignee: Streck, Inc., LaVista, NE (US)

(21) Appl. No.: 12/940,112

(22) Filed: Nov. 5, 2010

Related U.S. Application Data
(60) Provisional application No. 61/258,775, filed on Nov. 6, 2009.

(51) Int. Cl. A61K 39/12; C12N 7/06; C12Q 1/70; A61P 31/12; A61P 37/04

(52) U.S. Cl. 424/204.1; 435/238; 435/5

(57) ABSTRACT

The present invention is a composition comprising a live virus having an infectious component and a plurality of surface antigens in contact with a formaldehyde donor agent having a molecular weight that is less than about 400 g/mol. The present invention further provides a method for deactivating a live virus having an infectious component and a plurality of surface antigens, comprising the steps of: a) providing a live virus having an infectious component and a plurality of surface antigens; and b) contacting the virus with a formaldehyde donor agent having a molecular weight that is greater than about 50 g/mol and less than about 400 g/mol for a period of time (e.g., at least about 12 hours) sufficient for de-activating the infectious component with the formaldehyde donor agent and for preserving at least a portion of the surface antigens to form a deactivated virus. In another embodiment the invention is a method of preparing a composition useful as a vaccine comprising the abovementioned steps in combination with the step of c) mixing a non-toxic effective amount, for inducing an immune response in a subject to which the vaccine is administered, of the deactivated virus with a pharmaceutically acceptable carrier. Preferably the composition containing a pharmaceutically acceptable carrier is useful in, or as, a vaccine composition.

### CLAIM OF PRIORITY

This application claims the benefit of the filing date of U.S. Provisional Application No. 61/258,775 filed Nov. 6, 2009.

### FIELD OF THE INVENTION

The present invention relates generally to inactivated virus compositions, methods of preparing such compositions, and to compositions useful as or in vaccines.

### BACKGROUND OF THE INVENTION

It is known that the preparation of vaccines commonly involve the steps of inactivating a live virus (whether it be attenuated, modified or killed), so that the virus thereafter can be introduced into a living being for inducing an active immune response (e.g., a protective response) in the being. The manufacture of such vaccines often encounters any of a number of practical constraints, which create a strain on health care systems. By way of example, in recent history, shortages of influenza vaccines have been experienced due to contamination problems in the manufacturing process.

In the context of viral vaccines, one approach to the manufacture of such vaccines has been to grow the specific virus in advance of preparing the vaccine composition, such as by growing the virus in chicken eggs. The manufacture of viruses according to that approach can be expensive, and time consuming, especially considering that many times, one or two eggs may be necessary to yield each dose of vaccine. One classic approach to the manufacture of vaccines has been to inactivate a virus with formalin, binary ethylenimine, formaldehyde or combinations thereof. It is also typically necessary in the manufacture of vaccines to employ one or more preservatives to help prevent contamination by bacteria, fungus or both. Among the most widely employed preservatives have been phenol, 2-phenoxyethanol, or thimerosal (which contains mercury). Concerns have been expressed as to the potential efficancy, toxicity or mutagenicity of one or more of the above agents or other ingredients of vaccines. There has been a recognized resurgence in vaccine research in recent years, which has placed an even more acute demand upon vaccine manufacturers to address the above issues.

Newcastle Disease Virus (NDV) is a type of paramyxovirus. Attempts to address Newcastle disease by immunization are illustrated by published U.S. Patent Application Nos. 20070077559; 20070178115; 20030026813; U.S. Pat. No. 7,332,169; 7,252,984; 5,427,791; 4,235,876; and 4,053,583; all incorporated herein by reference. Protection of chickens against overt clinical disease and determination of viral shedding following vaccination with commercially available Newcastle disease virus vaccines upon challenge with highly virulent virus from the California 2002 exotic Newcastle disease outbreak is desired. The United States Department of Agriculture has approved protocols for using binary ethylene-imine of formaldehyde to inactivate Newcastle Disease Virus for vaccine production. In U.S. Pat. Nos. 5,459,073; 5,811,099; and 5,849,517, there are disclosed preservative compositions that include a formaldehyde donor agent, and specifically, one or both of diazolidinyl urea or imidazolidinyl urea. That such compositions can be used in a vaccine is addressed in U.S. Pat. Nos. 5,811,099; and 5,849,517.

Notwithstanding the foregoing, there remains a need in the art for safer and less toxic inactivated live virus compositions useful in vaccines, more specifically, veterinary vaccines, and still more specifically, inactivated-viral avian vaccines, which can be produced in high yield, with inconsequential toxicity or other potential undesired side effects.

### SUMMARY OF THE INVENTION

The present invention is directed at a composition comprising a live virus having an infectious component and a plurality of surface antigens in contact with a formaldehyde donor agent having a molecular weight that is less than about 400 g/mol. Preferably, the formaldehyde donor agent is selected from a non-crosslinking chemical fixative that contains urea.

The present invention provides a method for deactivating a live virus having an infectious component and a plurality of surface antigens, comprising the steps of: a) providing a live virus having an infectious component and a plurality of surface antigens; and b) contacting the virus with a formaldehyde donor agent having a molecular weight that is greater than about 50 g/mol and less than about 400 g/mol for a period of time (e.g., at least about 12 hours) sufficient for de-activating the infectious component with the formaldehyde donor agent and for preserving at least a portion of the surface antigens to form a deactivated virus. In another embodiment, the invention is a method of preparing a composition useful as a vaccine comprising the abovementioned steps in combination with the step of c) mixing a non-toxic effective amount, for inducing an immune response in a subject to which the vaccine is administered, of the deactivated virus with a pharmaceutically acceptable carrier. Preferably the composition containing a pharmaceutically acceptable carrier is useful in, or as, a vaccine composition.

The invention herein also contemplates vaccine compositions prepared according to the methods or using the virus of the foregoing aspects of the invention. The aspects of the present invention offer a number of advantages as compared with existing compositions useful in or as vaccines including the possibility to obtain total protection of mortality without use of an adjuvant; the ability to withstand without damaging the vaccine and/or without aggregating vaccine components after one or more freeze-thaw cycles; a reduction of cross-linking surface antigens to a significant enough degree that would result in an auto-immune hypersensitivity reaction mediated by mast cells is avoided; the vaccine protects birds from a live virus challenge (e.g., birds are given two vaccinations two weeks apart, and hemagglutination inhibition (HAI) data from samples drawn after the first vaccination indicate that a single vaccination is sufficient); a substantial reduction in the amount of antigen (e.g., a reduction by at least two logs in antigen, and more preferably at least three logs in antigen) necessary to manufacture the vaccine may be realized, a very important economic consideration.

### DETAILED DESCRIPTION OF THE INVENTION

As indicated, the present invention is directed at a composition comprising a live virus having an infectious component and a plurality of surface antigens in contact with a formaldehyde donor agent having a molecular weight that is less than about 400 g/mol. In addition the present invention provides an improved method for deactivating a live virus having an infectious component and a plurality of surface antigens, comprising the steps of: a) providing a live virus having an infectious component and a plurality of surface antigens; b) contacting the virus with a formaldehyde donor agent having a molecular weight that is greater than about 50 g/mol and less than about 400 g/mol for a period of time sufficient for de-activating the infectious component with the formaldehyde donor agent, and for preserving at least a portion of the surface antigens to form a deactivated virus. In another embodiment the invention is a method of preparing a composition comprising the abovementioned steps in combination with the step of c) mixing a non-toxic effective amount, for inducing an immune response in a subject to which the vaccine is administered, of the deactivated virus with a pharmaceutically acceptable carrier. Preferably the composition containing a pharmaceutically acceptable carrier is useful in, or as, a vaccine composition.

The amount of time for the contacting step preferably is sufficient for deactivating the virus (whether by attenuating it, modifying it or killing it), while sufficiently preserving surface antigens so that when a subject is vaccinated with a vaccine composition including the deactivated virus, the deactivated virus will induce an immunity response (e.g., a response that is measurable by Hemagglutination Inhibition Assay, such that the response allows the vaccinated subjects to survive exposure to a lethal dosage of the live virus).

In a preferred embodiment, the contacting occurs for a period of time of at least about 12 hours. For example, the contacting step occurs for a period of about 24 to about 72 hours. In a preferred embodiment, the contacting step occurs at a temperature of about 23° C. to about 37° C.; e.g., the contacting step (b) occurs at a temperature of about 25° C.

The live virus useful in this invention is any live virus having an infectious component and a plurality of surface antigens which is capable of being deactivated for use in vaccines. Preferred live viruses are those which are incubated in chicken eggs.

The viral infection that is useful in the invention herein preferably is an avian virus, and more specifically, the virus is Newcastle's disease. Still more specifically, the virus is a strain of Newcastle Disease Virus selected from a lentogenic strain, a mesogenic strain, a velogenic strain or any combination thereof. For example, the virus is a La Sota strain (e.g., Mass. type) of Newcastle Disease Virus.

When the virus is used in a composition used as a vaccine it is provided in a sufficient amount for invoking an immunity response in a subject to which the vaccines herein are administered. By way of example, the avian virus may be provided in a live titer amount of about 10⁶ to about 10¹¹ antigen per milliliter of the resulting vaccine composition. More specifically, the virus titer may range from about 2-4×10⁸ to about 2-4×10¹⁰.

The formaldehyde donor agent is selected from a non-crosslinking chemical fixative that contains urea. More preferably, the formaldehyde donor agent is selected from diazolidinyl urea (DU), imidazolidinyl urea (IDU), or a mixture thereof. The formaldehyde donor agent may consist essentially of diazolidinyl urea (DU) imidazolidinyl urea (IDU), or a mixture thereof.

The contacting step generally includes contacting the virus with the formaldehyde donor agent having a concentration of about 5 w/v (grams per 100 ml total volume) or less. Preferably, the formaldehyde donor agent may be present in a concentration of about 0.5 w/v (grams per 100 ml total volume) to about 5 w/v (grams per 100 ml total volume), in a solution during the contacting step. The formaldehyde donor agent may be present in a concentration of less than 5 w/v (grams per 100 ml total volume), in a solution during the contacting step. The formaldehyde donor agent may be present in a concentration of about 1 w/v (grams per 100 ml total volume) to about 4 w/v (grams per 100 ml total volume), in a solution during the contacting step. The contacting step may be the only step during which the live virus is contacted with the formaldehyde donor agent.

The mixing step is a step that can be employed for preparing the resulting composition useful as a vaccine. The mixing step may occur immediately following the contacting step (such that there is no intermediate washing, quenching step or other step by which the deactivated virus would be subjected to a fluid other than a carrier of the vaccine composition), or it may occur during the time period when the contacting step (b) is occurring. Thus, it will be appreciated that during the mixing step the deactivated virus will remain in contact with solution that includes the formaldehyde donor agent. During the mixing step, the antigen-treated virus may be maintained at a temperature above about 15° C. (e.g., it may be about room temperature). The composition of the invention generally will thus include a pharmaceutically acceptable carrier or diluent, which may be a liquid, a solid, a gel or otherwise.

By way of illustration, the antigen-treated virus may be admixed in a suitable carrier (e.g., water or saline) that optionally is buffered (e.g., phosphate buffered saline, such as Dulbecco's phosphate buffered saline “D-PBS”) before administering into a subject animal. Preferably, the carrier is such that the antigen-treated virus is uniformly dispersed in the resulting composition at the time of the administration, and it will not degrade the antigen-treated virus throughout a storage life of at least 10 days, more preferably at least one month at a temperature of about 0° to about 37° C. An example of one suitable solution includes a mixture of CaCl₂ (Calcium Chloride); MgCl₂ (Magnesium Chloride); KCl (Potassium Chloride); KH₂PO₄ (Potassium Phosphate, monobasic); NaCl (Sodium Chloride); Na₂HPO₄ (Sodium Phosphate, dibasic);and D-Glucose (dextrose). More specifically, one example of such a solution is set forth in the following Table 1, where mM refers to milli-molarity; millimoles/liter.

TABLE I

| | | |
|---|---|---|
| CaCl₂ | 0.901 | mM |
| MgCl₂ | 0.493 | mM |
| KCl | 2.67 | mM |
| KH₂PO₄ | 1.47 | mM |
| NaCl | 137.93 | mM |
| Na₂HPO₄ | 8.06 | mM |
| D-Glucose | 5.56 | mM |

As taught in U.S. Pat. No. 7,252,984, a carrier or diluent may include one or any combination of stabilizers, preservatives and buffers. Suitable stabilizers may include, for example SPGA, carbohydrates (such as sorbitol, mannitol, starch, sucrose, peptone, arginine, dextran, glutamate or glucose), proteins (such as dried milk serum, albumin or casein) or degradation products thereof. Suitable buffers may include for example alkali metal phosphates. Suitable preservatives may include thimerosal, merthuilate and gentamicin. Diluents include water, aqueous buffer (such as buffered saline) and polyols (such as glycerol). It will be appreciated that vaccine compositions herein, as well as any of its carrier or diluents is preferably free of any anti-biotic, and/or any mercury-containing ingredient.

The method may further comprise a step of contacting the virus with an adjuvant, such as an adjuvant selected from one or any combination of lecithin, a pharmaceutically acceptable polymer, saponin or a derivative thereof, or cholesterol.

If an adjuvant is employed, it may be employed in a suitable amount for providing adjuvant activity. Thus, by way of example, following treatment of the surface antigens of the virus, the deactivated virus may diluted into D-PBS, which may include an adjuvant or be free of an adjuvant. The adjuvant may include one or any combination of lecithin, a pharmaceutically acceptable polymer, saponin or a derivative thereof, or cholesterol. The total concentration of the adjuvant in the resulting final vaccine composition, when employed may range from about 50 μg/ml to about 7000 μg/ml, more preferably about 100 μg/ml to about 5500 μg/ml.

By way of example the individual components of the adjuvant may range (in the resulting composition) from about 2000 to about 4000, and more preferably about 3000 μg/ml Lecithin; about 1000 to about 3000 and more preferably about 2000 μg/ml of the pharmaceutically acceptable polymer (e.g., a Carboxypolymethylene, such as Carbopol® 974PNF, from Lubrizol); about 50 to about 200, and more preferably about 125 μg/ml of the saponin or a derivative thereof (e.g., Quil A); or about 50 to about 200, and more preferably about 125 μg/ml of cholesterol.

Other examples of suitable adjuvants may include one or any combination of compounds or compositions for this purpose include aluminium hydroxide, -phosphate or -oxide, oil-in-water or water-in-oil emulsion based on, for example a mineral oil, a vegetable oil such as vitamin E acetate, or some other suitable carboxylic acid. See, e.g., U.S. Pat. No. 5,152,981, incorporated by reference.

The composition and method for making it is a relatively simple and elegant to a technology that previously has seen various potentially complicating approaches. Though such approaches may be employed with the teachings herein, it is preferable that the vaccine composition and the virus are free of any contact with any plant-cell-produced components; the virus includes or is free of an infectious genetically engineered, genetically modified or cloned virus; the method may optionally be free of any step of contacting the virus with binary ethylene-imine, formaldehyde, formalin, phenol, 2-phenoxyethanol, thimerosal, bromo-ethylene-imine, ethyl methane sulfonate, nitrosoguanidine, fluorouracil, 5-azacytadine, or any combination thereof; or any combination of the foregoing. It is also possible that the vaccine composition may include or be free of any neutralizing antibodies bound to the surface antigens. It is also possible that the vaccine composition may otherwise include or be free of an anti-oxidant.

Other variations of the invention are also possible. To illustrate, the method may include a step of freeze-drying and re-hydrating the antigen-treated virus. The antigen-treated virus may include or be free of any temperature reduction to a temperature below about 10° C. (e.g., to below about 0° C.). The method may include or be free of any step that removes the formaldehyde donor agent prior to the mixing step. Accordingly, the various aspects of the invention contemplate that the administration of the vaccine composition is accomplished in the absence of any step of diluting the composition at the time of or at the site of administration of the vaccination. The mixing step described previously could constitute the only diluting step that occurs in the methods herein.

The method may also include one or more steps of storing the resulting composition for a period of at least 30 days, at least 60 days, or even at least 90 days following the mixing step; storing the resulting composition at one or more temperatures of below about 15° C. (e.g., about 0° to about 10° C.), one or more temperatures of above 15° C. (e.g., about 18° to about 30° C.);, or any combination thereof, with the composition retaining its stability under such conditions (e.g., the amount of useful deactivated virus remains within about 20% and more preferably within about 10% of the titer amount at the time of the mixing step). The composition may be stored in suitable sealed containers, such as stopped vials (e.g., rubber stopped glass or plastic vials), sealed syringes, blister packaging, or otherwise.

The method may also include one or more steps of performing an assay of the deactivated virus to confirm that the infectious component has been de-activated.

The methods herein further contemplate immunizing a subject with a vaccine composition prepared according to the present teachings. Thus the methods herein may further comprise at least one step of administering a subject in need of immunization with the vaccine composition described in the present teachings. Any step of administering a subject in need of immunization with the vaccine composition by performed by ingestion (e.g., from drinking water), intranasally (e.g., by aerosol), intraocularly (e.g., by aerosol), via intramuscular injection, by subcutaneous injection, by delivering into eggs prior to hatching (e.g., manually or by machine); or any combination thereof.

A plurality of steps of administering the vaccine to the subject may be performed at intervals (e.g., they may be administered to the same subject on multiple occasions, such as at intervals of at least 3 days). By way of further example, at least two steps of administering the subject may performed at intervals of greater than one week (e.g., about 14 days apart). It is also possible that only a single dose is administered and is sufficient for achieving the desired satisfactory immune response.

As gathered from the foregoing, another aspect of the present invention is directed at deactivated virus described herein that has been contacted with a formaldehyde donor agent according to the above teachings, as well as compositions useful as vaccines (including the and one or more of the other ingredients, e.g., one or more of a carrier, an adjuvant, or both) resulting from the methods herein. Resulting viruses treated according to the teachings herein may be characterized as being free of cross-linking with other surface antigens of the virus.

The dosage of the vaccine preferably will be a sufficient amount for inducing immunity in the vaccinated subjects against challenge by a virulent form of the virus, wherein immunity can be described as the realization within a period of 4, days, more preferably 7 days, and still more preferably 14 days after challenge of a death rate due to the virulent form of the virus that is less than 50% of the population of subjects challenged, more preferably less than 25% of subjects challenged and still more preferably less than 5% of subjects challenged (e.g., approximately 100% of challenged subjects survive the challenge). Challenges include the step of administering the virulent form of the virus to the subject in a 100% lethal dosage amount.

The strain of Newcastle Disease virus useful as a starting material (i.e., prior to any contact with a formaldehyde donor agent) may be selected from a lentogenic strain, a mesogenic strain, a velogenic strain or any combination thereof. It may be a genetically engineered strain, a genetically modified strain, a clone of an existing strain or any combination thereof. The vaccine compositions may be a combination vaccine that also includes a live vector virus. See e.g., U.S. Pat. No. 5,733,556, incorporated by reference.

Though illustrated in connection with immunization for prevention of Newcastle Disease Virus, the present invention is not intended to be so limited. It may have application for immunization for prevention of other viruses, including but not limited to other avian paramyxoviruses, avian influenza (e.g., H5 or H7 influenza, such as H5N1 influenza), avian polyoma virus, Pacheco's disease, West Nile Virus, diminuvirus, chicken anemia virus, or Circo virus. The vaccine compositions herein may also include a plurality of antigenic components suitable for immunizing against a plurality of viruses. For example, the invention contemplates that the resulting vaccine may immunize against Newcastle Disease Virus and one or more other viruses.

The methods and compositions herein may be employed in the immunization of mammal and avian subjects. Though preferably they are employed for immunizing chickens, they may be employed for other avian species as well.

The method of the invention comprises the steps of:
providing a live virus having an infectious component and a plurality of surface antigens; b) contacting the virus with a formaldehyde donor agent having a molecular weight that is greater than about 50 g/mol and less than about 400 g/mol for a period of time sufficient for de-activating the infectious component with the formaldehyde donor agent, and for preserving at least a portion of the surface antigens to form a deactivated virus. The method of the invention may further comprise one or more of the following features in any combination: the virus is grown in a chicken egg; the virus is Newcastle's disease; the virus is a strain of Newcastle Disease Virus selected from a lentogenic strain, a mesogenic strain, a velogenic strain or any combination thereof; the virus is a La Sota strain (e.g., Mass. type) of Newcastle Disease Virus; the formaldehyde donor agent is selected, or consists essentially of, from a non-crosslinking chemical fixative that contains urea; the formaldehyde donor agent is selected from, or consists essentially of, diazolidinyl urea (DU), imidazolidinyl urea (IDU), or a mixture thereof; the resulting solution consists essentially of de-activated virus and the formaldehyde donor agent; the contacting step includes contacting the virus with the formaldehyde donor agent having a concentration of about 5 w/v (grams per 100 ml total volume) or less, preferably about 0.5 w/v (grams per 100 ml total volume) to about 5 w/v (grams per 100 ml total volume), and more preferably about of about 1 w/v (grams per 100 ml total volume) to about 4 w/v (grams per 100 ml total volume); the contacting step (b) occurs for a period of about 24 to about 72 hours; wherein the contacting step (b) occurs at a temperature of about 23° C. to about 37° C., preferably about 25° C.; the mixing step (c) occurs immediately following the contacting step (b); during the time period throughout the contacting step (b) the antigen-treated virus is maintained at a temperature above about 15° C.; the contacting step (b) is the only step during which the live virus is contacted with the formaldehyde donor agent; the resulting composition is free of any contact with any plant-cell-produced components;, the virus includes an infectious genetically engineered, genetically modified or cloned virus; the virus is free of any infectious genetically engineered, genetically modified or cloned virus; the method is free of any step of contacting the virus with binary ethylene-imine, formaldehyde, formalin, phenol, 2-phenoxyethanol, thimerosal, bromo-ethylene-imine, ethyl methane sulfonate, Nitrosoguanidine, fluorouracil, 5-azacytadine, or any combination thereof; the method includes a step of freeze-drying and re-hydrating the antigen-treated virus; further comprising a step of performing an assay of the deactivated virus to confirm that the infectious component has been de-activated; the method is free of any step of reducing the temperature of the virus to below about 0° C.; the surface antigens are free of cross-linking with other surface antigens of the virus; the method further comprises; c) mixing a non-toxic effective amount for inducing an immune response in a subject to which the vaccine is administered of the deactivated virus with a pharmaceutically acceptable carrier for forming a vaccine composition; the virus is an avian virus provided in a live titer amount of about 10⁶ to about 10¹¹ EID₅₀ per milliliter of the resulting vaccine composition; the method further comprises a step of contacting the virus with an adjuvant; the adjuvant is selected from one or any combination of lecithin, a pharmaceutically acceptable polymer, saponin or a derivative thereof, or cholesterol; the mixing step (c) occurs immediately following the contacting step (b); during the time period throughout the contacting step (b) and the mixing step (c), the antigen-treated virus is maintained at a temperature above about 15° C.; during the time period throughout the contacting step (b) and the mixing step (c), the antigen-treated virus is free of any temperature reduction to a temperature below about 10° C.; and the method is free of any step that removes the formaldehyde donor agent prior to the mixing step (c).

The composition of the invention comprises a deactivated virus having an infectious component and a plurality of surface antigens in contact with a formaldehyde donor agent having a molecular weight that is less than about 400 g/mol. The composition of the invention may further comprise one or more of the following features in any combination: wherein the virus manufactured by growing the virus in a chicken egg; the virus is a live Newcastle Disease Virus; the virus is a strain of Newcastle Disease Virus selected from a lentogenic strain, a mesogenic strain, a velogenic strain or any combination thereof; the virus is a La Sota strain (e.g., Mass. type) of Newcastle Disease Virus; the formaldehyde donor agent is selected, consists essentially of, from a non-crosslinking chemical fixative that contains urea; the formaldehyde donor agent is selected from, consists essentially of, diazolidinyl urea (DU), imidazolidinyl urea (IDU), or a mixture thereof the formaldehyde donor agent is present in a concentration of about 5 w/v (grams per 100 ml total volume) or less; the formaldehyde donor agent consists essentially of diazolidinyl urea (IDU), preferably about 0.5 w/v (grams per 100 ml total volume) to about 5 w/v (grams per 100 ml total volume); the composition further comprises a pharmaceutically acceptable carrier or diluent, and more preferably about of about 1 w/v (grams per 100 ml total volume) to about 4 w/v (grams per 100 ml total volume); the resulting composition is free of any contact with any plant-cell-produced components; the virus includes an infectious genetically engineered, genetically modified or cloned virus; the virus is free of any infectious genetically engineered, genetically modified or cloned virus; the composition is free of binary ethylene-imine, formaldehyde, formalin, phenol, 2-phenoxyethanol, thimerosal, bromo-ethylene-imine, ethyl methane sulfonate, Nitrosoguanidine, fluorouracil, 5-azacytadine, or any combination thereof; the surface antigens are free of cross-linking with other surface antigens of the virus; a non-toxic effective amount for inducing an immune response in a subject to which the vaccine is administered of the deactivated virus of a pharmaceutically acceptable carrier; the virus is an avian virus provided in a live titer amount of about 10⁶ to about 10¹¹ EID₅₀ per milliliter; an adjuvant; and an adjuvant is selected from one or any combination of lecithin, a pharmaceutically acceptable polymer, saponin or a derivative thereof, or cholesterol.

### EXAMPLES

### Example 1

Chicken embryo toxicity by DU and IDU solutions (no virus) is tested. IDU and DU solutions (1%, 3%, 5% in PBS) are injected in an amount of about 100 μl into the allantoic fluid of 7 day old chicken embryos and the embryos are monitored daily for five days. It is expected that this amount of the 5% DU solution is toxic to embryos, such that the embryos will show 0% survival, but 1%, 3%, DU and 1%, 3%, 5% IDU solutions are well tolerated by the embryos, with 100% survival.

### Example 2

The solutions of Example 1 for which 100% survival is expected are contacted with live La Sota strain of Newcastle Disease Virus and maintained in contacted at the temperatures of 2-7° C., 25° C., or 37° C., and for the times of 0 hour, 24 hour, or 72 hour. Commercial binary ethyleneimine (BEI) is used as a positive control for NDV inactivation, and PBS is used alone as a negative control for NDV inactivation.

NDV inactivation and surface epitope integrity are determined by some or all of the following assays.

Inactivation Sample Hemagglutination Assay (HA) is run to determine if inactivation treatment degrades NDV surface epitopes necessary for hemagglutination. If so, the HA result should be negative. The desired result is positive hemagglutination, thereby confirming that surface epitopes are preserved. More specifically, in this assay, a sample is incubated with a fresh preparation of chicken red blood cells (cRBCs). Hemagglutination (HA) is defined as the clumping of cRBCs. NDV can promote hemagglutination through the interaction of molecules on the virus surface with molecules on the surface of cRBCs. Damaged or degraded virus does not promote hemagglutination. Depending on the sample used (treated/untreated virus or allantoic fluid—AF—from inoculated eggs), the results may be interpreted differently. If the sample is treated/untreated virus (the inactivation sample), a positive HA result indicates that the virus is not degraded due to the treatment—it gives no information (in this case) about the viability of the virus (inactivation state). If the sample is the allantoic fluid from an inoculated egg, a positive HA result indicates that the virus is not inactivated by the treatment (the virus was active and able to replicate in the egg). If the virus is completely inactivated by the treatment (eg—incubation with DU/IDU/BEI/formaldehyde), the small amount of virus in the inoculum would be diluted out by the large volume of AF, and the HA result would be negative.

Embryonic Toxicity assay is run to determine if the virus is still viable after the inactivation treatment. If still viable the virus would replicate in allantoic fluid and kill an embryo to which it is contacted. The desired result is embryonic viability, thereby confirming the presence of no live virus. More specifically, embryonic toxicity is assessed by candling the eggs, such that an egg is placed on a source of bright light (e.g. a flashlight), and the embryo is directly observed. Viable embryos are identified by certain visual cues (pulsatile vasculature, for example). In these experiments, embryonic death may be due to one of three things: (1) injury/infection resulting from the needle stick used in the injection; (2) chemical toxicity (by inactivating agent—DU/IDU); or (3) live virus replication (as a result of incomplete NDV inactivation).

Allantoic fluid (AF; from infected eggs) HA Assay is run to determine if NDV not viable. If not viable, inactivated NDV would not replicate and injected virus would be diluted out in AF. If, after ‘inactivation’ treatment, NDV still viable, allantoic fluid would contain high enough titer of virus to promote hemagglutination. The desired result is negative hemagglutination, thereby confirming that the AF fluid contains no live virus.

Chicken Embryonic Fibroblast (CEF) Immunofluorescence (IF) Assay is run to determine whether successful inactivation occurs. It is recognized that chicken embryonic fibroblasts (CEF) grow well in culture and are highly susceptible to infection by live NDV. In this assay, allantoic fluid (AF) from inoculated eggs is applied to confluent CEF monolayers. After 5 days, cells are washed, fixed and stained with anti-NDV antibodies. Positive staining indicates that there was live virus in AF, and therefore, ‘inactivation’ treatment is unsuccessful. The desired result is no staining (which indicates that the virus had been completely inactivated), and supports that the AF fluid contains no live virus.

Hemagglutination Inhibition Assay is run with serum samples obtained from vaccinated and/or control animals are incubated with live NDV prior to use in the aforementioned HA assay. If the cRBCs do not agglutinate (negative result), the conclusion is that there were specific anti-NDV antibodies in the serum (seroconversion) which blocked the viral antigens from interacting with the cRBCs. A serum HAI titer of greater than 16 correlates with an animal successfully rejecting a subsequent live NDV challenge. An example of one such assay is found in Allan and Gough, “A standard haemagglutination inhibition test for Newcastle disease. (2) Vaccination and challenge”. Vet Rec., Vol. 95, Issue 7, 147-149, Aug. 17, 1974.

TABLE 1

| Sample | DU/IDU | Concentration | time | temperature |
|---|---|---|---|---|
| 1 | IDU | 1% | 72 hour | 25° C. |
| 2 | IDU | 3% | 72 hour | 25° C. |
| 3 | IDU | 5% | 72 hour | 25° C. |
| 4 | IDU | 1% | 24 hour | 25° C. |
| 5 | IDU | 3% | 24 hour | 25° C. |
| 6 | IDU | 5% | 24 hour | 25° C. |
| 7 | DU | 1% | 24 hour | 25° C. |

Example 3

Samples 1 through 7 are subjected to the same treatment as in Example 2 and are ranked for identifying the top four candidates. Samples 1, 2, 3 and 5 are selected for further study. Vaccines are prepared for each NDV inactivation sample. Vaccines are also prepared for samples inactivated by binary ethylene-imine (BEI) and formalin as comparison. The vaccine is prepared with standard adjuvants, but in addition to those, straight inactivation sample diluted in PBS was used as ‘un-adjuvanted’ vaccines.

Blood samples are harvested from naïve birds prior to vaccination (pre-bleed). Birds are vaccinated twice—once immediately after the ‘pre-bleed’ and again two weeks later. Blood samples are obtained two weeks after each vaccination. After the second post-vaccination blood sample is obtained, birds are challenged with an inoculation of live NDV virus.

Vaccine efficacy is determined by two readouts. First, Hemagglutination Inhibition (HAI) Assay is run to determine if the vaccination results in the presence of circulating anti-NDV antibodies. If so, mixing live NDV with serum from vaccinated birds should prevent the virus from hemagglutinating chicken RBCs. The desired result is measurable inhibition of hemagglutination in the standard HA assay (high GMT, or geometric mean titer). Next, viability of birds challenged with live virus is analyzed to determine if birds survive live NDV challenge. The desired result is survival (protection), which would indicate a successful vaccination.

All vaccines prepared from IDU-inactivated NDV successfully protect birds from live virus challenge. The ‘un-adjuvanted’ vaccines protect birds equally as well as adjuvanted vaccines (although the GMT in the HAI assays may be lower for un-adjuvanted vs adjuvanted vaccines).

The measurement of virus in a suspension is measured as set forth in Grimes, “A Basic Laboratory Manual for the Small-Scale Production and Testing of 1-2 Newcastle Disease Vaccine” (2002), RAP Publication 2002/22 (ISBN 974-7946-26-2), incorporated by reference.

For all teachings in herein, any numerical values recited include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight.

Unless otherwise stated, all ranges include both endpoints and all numbers between the endpoints. The use of “about” or “approximately” in connection with a range applies to both ends of the range. Thus, “about 10 to 20” is intended to cover “about 10 to about 20”, inclusive of at least the specified endpoints.

The disclosures of all articles and references, including patent applications and publications, are incorporated by reference for all purposes.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

What is claimed is:

1) A method comprising the steps of:
a) providing a live virus having an infectious component and a plurality of surface antigens;
b) contacting the virus with a formaldehyde donor agent having a molecular weight that is greater than about 50 g/mol and less than about 400 g/mol for a period of time sufficient for de-activating the infectious component with the formaldehyde donor agent, and for preserving at least a portion of the surface antigens to form a deactivated virus.

2) The method of claim 1 wherein the virus is grown in a chicken egg.

3) The method of claim 1 wherein the formaldehyde donor agent is selected from a non-crosslinking chemical fixative that contains urea.

4) The method of claims 3 wherein the formaldehyde donor agent is selected from diazolidinyl urea (DU), imidazolidinyl urea (IDU), or a mixture thereof.

5) The method of claim 1 wherein the contacting step includes contacting the virus with the formaldehyde donor agent having a concentration of about 5 w/v (grams per 100 ml total volume) or less.

6) The method of claim 1 wherein the contacting step (b) occurs for a period of about 24 to about 72 hours.

7) The method of claim 1 wherein the contacting step (b) occurs at a temperature of about 23° C to about 37° C.

8) The method of claim 1 wherein the method is free of any step of contacting the virus with binary ethylene-imine, formaldehyde, formalin, phenol, 2-phenoxyethanol, thimerosal, bromo-ethylene-imine, ethyl methane sulfonate, Nitrosoguanidine, fluorouracil, 5-azacytadine, or any combination thereof.

9) The method of claims 1 wherein the method includes a step of freeze-drying and re-hydrating the antigen-treated virus.

10) The method of claim 1 further comprising a step of performing an assay of the deactivated virus to confirm that the infectious component has been de-activated.

11) The method of preparing a vaccine comprising the method of claim 1 which further comprises; c) mixing a non-toxic effective amount for inducing an immune response in a subject to which the vaccine is administered of the deactivated virus with a pharmaceutically acceptable carrier for forming a vaccine composition.

12) The method of claim 11 wherein the virus is an avian virus provided in a live titer amount of about 10⁶ to about 10¹¹ EID₅₀ per milliliter of the resulting vaccine composition.

13) The method of claims 11 further comprising a step of contacting the virus with an adjuvant.

14) The method of claims 10 wherein the mixing step (c) occurs immediately following the contacting step (b).

15) A composition comprising a deactivated virus having an infectious component and a plurality of surface antigens in contact with a formaldehyde donor agent having a molecular weight that is less than about 400 g/mol.

16) The composition of claim 15, wherein the virus manufactured by growing the virus in a chicken egg.

17) The composition of claim 15 wherein the formaldehyde donor agent is selected from a non-crosslinking chemical fixative that contains urea.

18) The composition of claim 15 wherein the formaldehyde donor agent is selected from diazolidinyl urea (DU), imidazolidinyl urea (IDU), or a mixture thereof.

19. composition of claim 15 wherein the formaldehyde donor agent is present in a concentration of about 5 w/v (grams per 100 ml total volume) or less

20) The composition of claim 15 which further comprises a pharmaceutically acceptable carrier or diluent.
